(19) United States
(12) Patent Application Publication
(10) Pub. No.: US 2011/0020413 A1
(43) Pub. Date: Jan. 27, 2011

(54) POLYSILSESQUIOXANE COMPOSITIONS AND PROCESS

(76) Inventors: John GORMLEY, Midland Park, NJ (US); Steven Isaacman, New York, NY (US)

Correspondence Address: KF Ross PC, 5683 Riverdale Avenue, Box 900, Bronx, NY, 10471, US

(21) Appl. No.: 12/687,186

(22) Filed: Jan. 14, 2010

Related U.S. Application Data
(60) Provisional application No. 61/145,623, filed on Jan. 19, 2009.

(51) Int. Cl. A61K 8/89; A61Q 1/06; A61K 8/02; A61Q 19/10; A61Q 3/02; C08K 5/3445; A61Q 17/04; A61Q 19/08; A61Q 5/02; A61Q 5/06; A61Q 11/00; A61Q 5/12; A61Q 1/12; B32B 15/02; A61Q 1/10; A61P 17/16; A61P 17/02; C08L 83/04

(52) U.S. Cl. 424/401; 8/405; 424/49; 424/59; 424/61; 424/63; 424/64; 424/69; 424/70.6; 424/70.7; 424/70.121; 427/213.32; 428/407; 510/122; 510/130; 510/158; 524/588

(57) ABSTRACT

Composite particles comprised of an optical agent(s) entrapped in a non-porous microparticle of polyorganosilsesquioxane to yield discrete microparticles, are provided. The optical agent is fully encapsulated in the interior of the silsesquioxane particle to impart optical properties that include absorption and emission in the ultraviolet and visible spectrum respectively, without otherwise interfering with the surface chemistry or surface properties of the particle. The use of these novel microparticles in a cosmetic or pharmaceutical composition provides a change in the optical appearance of the skin by either changing the apparent tone or hue, making the skin appear lighter, softer, or by photoluminescent brightening and/or by novel color sculpting under various light conditions.

### CROSS REFERENCE TO RELATED APPLICATIONS

This application is related to co-pending U.S. Provisional Application Ser. No. 61/145,623 filed 19 Jan. 2009.

### FIELD OF THE INVENTION

The present invention relates to non-porous hydrophobic spherical microparticles of polymethylsilsesquioxane (PMSQ) containing optical agents fully entrapped therein, the synthesis of these particles and their use in cosmetic, pharmaceutical and other personal care compositions and methods of making and using these compositions.

### BACKGROUND OF THE INVENTION

Optical agents are useful in cosmetics through their ability to both change the appearance of the cosmetic preparation in which they are formulated, and to alter the appearance of the skin, hair or nails when applied. The utilization of optical methods to mask unwanted skin imperfections is crucial as it allows consumers an immediate, visual improvement. Emerett (Quantification of the Soft-Focus Effect, Cosmetics and Toiletries, Vol. 111. 7, 1996, pp 57-61) teaches that wrinkles and fine lines in human skin are primarily visible because light is not reflected out of them. Attempts to reduce the appearance of skin imperfections and alter the perceived hue of the skin have led to using colored substances to counteract the undesirable redness associated with older looking skin. WO 00/51551 teaches the use of low levels of green pigments applied to the skin to decrease the redness of the skin; however, the look achieved remains undesirable. In addition, pigments may inherently contain unfavorable sensory properties which are transferred to the delivery vehicle upon incorporation. Other attempts to incorporate optical agents include direct incorporation of approved FD&C and D&C dyes into either the oil or water phase of said preparations. While the sensory applications are not affected, the dyes used tend to be soluble in water or oil, and tend to bleed when applied to the skin. Thus there remains a need for an optical agent which can impart desirable optical properties to aged skin while retaining an excellent sensory profile.

Photoluminescent compounds represent a type of optical agent which is useful in cosmetics by their ability to diffuse and soften light to alter the appearance of skin imperfections. The appearance of said imperfections including, but not limited to wrinkles, are diminished as the compound's inherent optical properties manipulate light in a favorable manner. Many industrial-grade photoluminescent compounds, such as textile brighteners, are considered safe for use in their intended applications, including intermittent human contact which occurs from transfer of the brightener to the skin from a garment. However, such brightener agents are generally not considered appropriate for direct use in leave-on cosmetics, due to potential toxicity from residual by-products.

To circumvent these issues and to prepare a cosmetically acceptable brightener, U.S. Pat. No. 6,946,147, incorporated herein in its entirety by reference, discloses a composition and method of encapsulating brighteners into a particle using a four step (swell/dry/coat/crosslink) process whereby a swellable, porous polymer particle was infused with a volatile solvent system containing a non-volatile brightener, then dried to leave the brightener on the surface of the polymer matrix, then encapsulated with a secondary translucent polymer overcoat and cross linked with a multivalent agent to form a shell like barrier. This last step, as taught therein, is an essential embodiment towards retaining the brightener in the polymer core. This multi step approach adds complexity to the creation of an optically active particle and has the drawback of requiring the use of potentially toxic cross linking agents like glyoxyl or formalin/formaldehyde to form the outer coating. In actual practice, said cross-linked secondary coating may contain defects, resulting in a portion of the entrapped brightener to be released into common formulating environments such as hot water.

A related patent, U.S. Pat. No. 6,808,722, incorporated herein in its entirety by reference, teaches starting with a plurality of preformed substrate particles and post-fixing a fluorescent agent to create optical-activated fixed particles. In addition, such surface fixation changes the inherent properties of the substrate microparticles by physically and chemically altering the surface chemistry of said particles. The fluorescent compound is fixed to a plurality of pre-existing substrate particles by ionic, covalent, or hydrogen bonding, Van der Waals forces, or by strong or weak physio-chemical association. This method has the drawback that the plurality of substrate particles must be inherently capable of interacting chemically or associatively with said fluorescent compound. Unfortunately, some classes of substrate particles are made of hardened, non-polar compounds that are not amendable to a post-fixing process of either the swell/dry method or by any of the chemical or associative means listed within the invention scope. Examples of such particles may include, but are not limited to: thermosets; thermoplastics (particularly when in solvent systems in which they can not be swelled or fixed without first irreversibly damaging the particle and/or the brighter compound); and silicon containing particles, such as glass, silica, organosilicates and polyorganosilsesquioxanes. In such instances, the attempt at fixing the fluorescent compound on the substrate particles using the methods taught therein would fail, resulting in only a poor surface coating that would be readily and undesirably washed off in the intended applications.

Polyorganosilsesquioxanes are polymers of the empirical formula [RSiO_{3/2}]ₙ, made by condensing silanes of the empirical formula RSi(R′)₃, where R is generally H, substituted or unsubstituted C₁₋₄ lower alky group or aryl group including phenyl; R′ is —Cl or —OR. This class of polymers can form several distinct morphological structures depending on the reaction conditions and variable radicals employed. For instance, a sheet-like coating resin is useful in electronic coatings when R is H. When R is methyl, mono-dispersed spherical micronized particles can be created that have broad utility as a filler and friction modifier in cosmetic and industrial applications and is toxicologically safe for use in food contact applications. Polymethylsilsesquioxane (PMSQ) powders, especially spherical powders, are frequently used in cosmetic formulations to obtain the benefits of excellent skin sensory feel, light diffusing effect, smooth texture, anti-caking and water repellency. As a class of materials, these hybrid organo-inorganic particles provide an elevated refractive index compared to organic only particles. In comparison with other synthetic polymer powders, PMSQ powders have an excellent heat resistance up to 400° C. and a higher purity because the residual byproducts and monomers can be easily removed by drying at about 300° C., at which temperature most polymer powders are decomposed or discolored. Many methods have been proposed in the art for the preparation of PMSQ powders. Belgian Patent 572,412 disclosed a process in which ethyltrichlorosilane is hydrolytically condensed in water. U.S. Pat. No. 4,528,390 discloses a process to make spherical PMSQ powder in which methyltrimethoxysilane was hydrolyzed and condensed in an aqueous solution of ammonia followed by washing, drying and pulverizing. The condensation forms a particle which grows to the maximum size that can be dispersed by the reaction medium. The amount and size stably dispersed can be increased by the addition of additives like surfactants and polymeric dispersants added before and/or during the reaction. The precipitated PMSQ particle is readily recovered by filtration to ideally yield monodispersed particles, preferentially of about 3-6 microns in diameter. It is well known in the art of making monodispersed PMSQ that changing a single reaction parameter like temperature or the amount of an additive could cause profound deviation of particle size distribution or form a brittle sheet like morphology of no relative utility.

Precipitated monodispersed PMSQ particles are readily collected and washed to remove impurities. In this reaction stage they exist as hydrated beads, containing anywhere from about 0.01% to about 10% free —OH groups. Irreversible cross-linking and complete drying is achieved in a dehydration chamber, typically at elevated temperatures to provide a particle that is highly durable, non-porous and essentially non-swellable in pH neutral hot water. The particles are non-ionic and not subject to ionic association. Furthermore, their ability to form intermolecular bonds, via Van der Waal interactions with other chemical compounds, is extremely limited.

Two or more oligomers of silsesquioxane can be coupled or bridged by use of an R group that can be covalently reactive towards a divalent bridging radical. As such, the choice of a bridging molecule is nearly unlimited and may include optical agents. For example, US Pat. Application 20050123760 discloses a method of covalently attaching reactive fluorescent dyes to form bridged covalent photo-luminescent silsesquioxane nanoparticle colorants. Likewise, US Pat application 20080029739 discloses another method of making fluorescent colorants from nanoparticles of polyhedral oligomeric silsesquioxanes (POSS) by covalently modifying said POSS with anhydride containing chromophores to produced highly UV stable colorants. The morphology in this case could be either spherical or sheet-like when in a fully condensed stage, but is chemically defined by having some degree of organic UV colorant on the surface of the particle or sheet, such that it cannot be washed off without an additional chemical scouring step. The main drawback of a chemically surface modified silsesquioxane is a change in the surface characteristics of the particle that can drastically alter the sensory feel and wet-ability of the particle when used in cosmetics. The dye chemistry on the surface is also susceptible to oxidation or other chemical changes from contact with external environment that could alter the optical properties or form an undesirable chemical byproduct.

### OBJECTS OF THE INVENTION

It is therefore an object of the present invention to synthesize a photoluminescent particle, with enhanced optical attributes, in a one polymer system, thus removing the drawbacks of a secondary coating process and the inherent issues of multivalent cross linking agents.

It is another object of the invention to create particles of polyorganosilsesquioxanes with inherent photoluminescent properties, such that there is no requirement to use a plurality of non-optically active particles and subsequently fix a fluorescent compound to them.

It is another aim of this invention to produce both uncured photoluminescent particles of micron-sized polyorganosilsesquioxane with a noncovalently linked optical agent encapsulated in the interior portion of the silsesquioxane particle, but not present on the surface, such that the sensory properties are not altered from standard PMSQ.

It is another aim of the invention to produce cured photoluminescent particles of micron-sized polyorgano-silsesquioxane with a noncovalently linked or a covalently linked optical agent encapsulated in the interior portion of the silsesquioxane particle, but not present on the surface, such that the sensory properties are not altered from standard PMSQ.

It is another object of the invention to synthesize a series of colored polyorganosilsesquioxane microparticles, containing entrapped D&C or FD&C dyes, whose sensory attributes are identical to those found in unmodified microparticles of identical size and shape.

It is a further aim to compound the particles of the present invention into a new personal care composition that adds brightness, colors and vibrancy to improve the visual appearance of the skin or hair.

### SUMMARY OF THE INVENTION

The present invention relates to particles comprised of optical agent(s) entrapped in a microparticle of polyorganosilsesquioxane, their synthesis and the use of these microparticles in cosmetic and pharmaceutical preparations. The particles have a size ranged from 1-20 microns with a narrow particle size distribution. The spherical powder is synthesized using a process comprising: (1) hydrolyzing and condensing an alkyltrialkoxysilane in water in the presence of the optical agent, a suspending agent, a surface tension modifier and an acidic or basic catalyst to form water-dispersible spherical particles; (2) purifying the spherical particles by repeated washing; and (3) optionally drying the particles at elevated temperatures to cure them to a highly cross linked state and make them completely hydrophobic and non-porous. The entrapped optical agent is physically separated from the external environment and contained in an extremely durable microsphere particle. The resulting size, shape and optical properties of the microparticle can be tailored to application requirements by selecting an appropriate optical agent. Uncured particles contain anywhere between 0.1%-10% of free —OH groups and are water dispersible, thus allowing for optional incorporation into the aqueous phase of cosmetic preparations in cases where the optical agent(s) is hydrophobic in nature.

### DETAILED DESCRIPTION OF THE INVENTION

The following terms shall be used to describe the present invention. In instances where a term is not provided with a specific definition herein, the common definition of a term given by those of ordinary skill within the context of the term's use is to be used to describe the invention.

The term “optical agent” is used to describe a plurality of common moieties which can impart an optical effect by adsorbing and reflecting light in the visual and ultraviolet region. Examples include industrial dyes such as optical brighteners, fluorophores, food dyes, textile dyes, FD&C and D&C dyes, and photoactive nanodiamond or photoactive nanometer sized particles of amorphous carbon. Optical brighteners are a specific class of fluorophores that absorb UV light (200-400 nm) and emit blue light in the visible spectrum. Fluorophores are molecules that absorb light of higher energy and emit light of lower energy, and may contain excitation and emission spectra throughout the UV and visible regions. Examples of optical brighteners are molecules that include, but are not limited to, derivatives of stilbene, biphenyl, naphthalene and anthracene. Exemplary molecules may be found in the Kirk-Othmer Encyclopedia of Chemical Technology. Examples of fluorophores include, but are not limited to, fluorescein, rhodamine, Cy5 and their derivatives. When the optical agent is a food dye, textile dye or D&C dye, the absorbance is in the visible region (400-800 nm). Exemplary molecules may be found in the FD&C Handbook and are chosen accordingly by their chemical and spectral properties by a person skilled in the art.

The term “nanoscale” as defined herein is a particle less than about 100 nm in size. The term “nanodiamond” is used to describe a variety of nanoscale carbon materials which include, but are not limited to, diamond based materials at the nanoscale, including pure phase diamond particles, mixtures of amorphous carbon and other carbon based nanoparticles. The nanodiamonds are commercially available in a number of different sizes, preferably ranging from about 1 nm to about 100 nm. The commercially available nanodiamonds are commonly known as “Nanodiamonds” or “Carbon Based Quantum Dots” or “Crystalline Nanodiamond” or “Detonation Nanodiamond” or “Non-Detonation Nanodiamond” or “Carbon Nanoparticles”. The nanodiamonds are also commercially available with a number of different photoluminescent properties. The nanodiamonds are also commercially available with a number of different surface functionalities. The preferred class of nanodiamond is commonly referred to as ultrananocrystalline diamond, produced by detonation synthesis, with characteristic size of about 10 nm. Another preferred class of nanodiamond is commonly referred to as amorphous carbon. Both of these materials are commercially available and can be used when fully entrapped in the microparticle. The material is available in a number of different purity levels and can be produced on a large scale by a variety of techniques. These techniques include, but are not limited to, detonation synthesis and laser ablation of a carbon target. A person skilled in the art can choose a commercially available nanodiamond or amorphous carbon material which exhibits the proper size, surface functionality and photoluminescent properties for incorporation into the organosilsesquioxane microparticle.

The term “subject” is used to describe a human capable of advantageously using compositions according to the present invention. It may also include animals, such as cats, dogs or horses, when the composition is used to enhance the appearance of an animal's coat.

The term “personal care composition” is used to describe a chemical composition or product used for the purpose of cleansing, conditioning, grooming, beautifying, or otherwise enhancing the appearance of a subject. Personal care products include skin care products, cosmetic products, antiperspirants, deodorants, perfume, toiletries, soaps, bath oils, feminine care products, hair-care products, oral hygiene products, depilatories, shampoos, conditioners, hair straightening products and other hair care products, color cosmetics such as lipstick, creams, make-up, skin creams, lotions (preferably comprised of water-in-oil or oil-in-water emulsions), shave creams and gels, after-shave lotions and shave-conditioning compositions and sunscreen products, among numerous others. In preferred aspects, personal care compositions according to the present invention include make-up, lipstick, skin creams (to hide skin imperfections and/or to promote anti-wrinkling) and other leave on skin-care products.

The term “effective” is used to describe an amount of a component or composition which is used or is included in a formulation or composition within context, to produce an intended effect.

The term “emulsion”, “water-in-oil emulsion” or “oil-in-water emulsion” are used throughout the specification to describe certain personal care compositions according to the present invention. An “emulsion” according to the present invention is advantageously a cream or lotion (especially a skin cream or skin lotion) which is generally formed by the suspension of a very finely divided liquid, in this case water, in another liquid, in this case oil. In the present invention, an emulsion is formed when the water phase is compatibilized in the oil phase, such that the water phase becomes “hidden” within the oil phase. Alternatively, an emulsion also may be formed when the oil phase is compatibilized in the water phase, such that the oil phase is “hidden” within the water phase. The term emulsion is used to distinguish the present compositions from compositions which contain at least two visually distinct phases, i.e., an oil phase and a water phase. Emulsions can be used to provide a number of personal care formulations including skin creams, skin lotions, color cosmetics, conditioners and shampoo formulations.

The term “oil” is used throughout the specification to describe any of various lubricious, hydrophobic and combustible substances obtained from animal, vegetable and mineral matter. Oils for use in the present invention may include petroleum-based oil derivatives such as purified petrolatum and mineral oil. Petroleum-derived oils include aliphatic or wax-based oils, aromatic or asphalt-based oils and mixed base oils and may include relatively polar and non-polar oils. “Non-polar” oils are generally oils such as petrolatum or mineral oil or its derivatives which are hydrocarbons and are more hydrophobic (lipophilic) compared to oils such as esters, which may be referred to as “polar” oils. It is understood that within the class of oils, that the use of the terms “non-polar” and “polar” are relative within this very hydrophobic and lipophilic class, and all of the oils tend to be much more hydrophobic and lipophilic than the water phase which is used in the present invention.

In addition to the above-described oils, certain essential oils derived from plants such as volatile liquids derived from flowers, stems and leaves and other parts of the plant which may include terpenoids and other natural products including triglycerides may also be considered oils for purposes of the present invention.

Petrolatum (mineral fat, petroleum jelly or mineral jelly) and mineral oil products for use in the present invention may be obtained from a variety of suppliers. These products may range widely in viscosity and other physical and chemical characteristics such as molecular weight and purity. Preferred petrolatum and mineral oil for use in the present invention are those which exhibit significant utility in cosmetic and pharmaceutical products and are “cosmetically compatible”. Cosmetic grade oils are preferred oils for use in the present invention.

Additional oils for use in the present invention may include, for example, mono-, di- and tri-glycerides which may be natural or synthetic (derived from esterification of glycerol and at least one organic acid, saturated or unsaturated, such as butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others. In addition, a number of other oils may be used, including C₁₂, to C₃₀ (or higher) fatty esters (other than the glyceride esters, which are described above) or any other acceptable cosmetic emollient.

In certain embodiments, cyclical silicone oils may be used such as cyclotetrasiloxane (D4), cyclopentasiloxane (D5) and cyclohexasiloxane (D6). Also, linear silicone oils such as trisiloxanes, including but not limited to, heptamethylethyltrisiloxane and dimethicones may be used. Branched silicones are also useful, including but not limited to methyl trimethicone and phenyl trimethicone. Cross-linked silicone elastomer gels containing silicone or other oils are highly useful in the current invention. Preferred, are the class of silicone elastomer gels containing silicone and/or hydrocarbon oils. Examples include the Gransil series such as GCM-5 (Grant Industries Inc, Elmwood Park N.J.).

The present invention relates to novel microparticles comprised of an optical agent(s) entrapped in a microsphere of polyorganosilsesquioxanes. A further cosmetic composition is disclosed containing a hydrophobic, spherical powder of polyorganosilsesquioxane having a particle size ranged from 1-20 microns with a narrow particle size distribution. A preferred narrow particle size distribution for the spherical, hydrophobic silsesquioxane powder is as follows: 99% or more of the particles are within 1 to 20 microns and 70% or more of the particles are within ±30% of the mean value of the particle size. The preferred mean value is around 5 microns. The spherical powder is synthesized using a process comprising: (1) hydrolyzing and condensing an alkyltrialkoxysilane in water in the presence of the optical agent, a suspending agent, a surface tension modifier and an acidic or basic catalyst to form water-dispersible spherical particles; (2) purifying the spherical particles by repeated washing and (3) optionally drying the particles at elevated temperatures to cure them to a highly cross linked state and make them completely hydrophobic and non-porous. The entrapped optical agent is physically separated from the external environment and contained in an extremely durable microsphere particle. The resulting size, shape and optical properties of the microparticle can be tailored to application requirements by selecting an appropriate optical agent. Uncured particles contain anywhere between 0.1%-10% of free —OH groups and are water dispersible, thus allowing for optional incorporation into the aqueous phase of cosmetic preparations in cases where the optical agent(s) is essentially hydrophobic in nature.

The polyorganosilsesquioxane microparticle formed in this invention is a polymer of the empirical formula [RSiO_{3/2}]ₙ, made by condensing silane monomers of the empirical formula RSi(R′)₃ in excess water, where R is generally H, a substituted or unsubstituted C₁₋₄ lower alky group or aryl group including phenyl; R′ is —Cl or —OR, whereby R groups can be the same or different and n is an integer. Preferred R′ is —OR, where both R groups are methyl, thus providing methyltrimethoxysilane as the silane phase starting material. This material is preferred compared to when R′ is fully —Cl, whereby such trichlorosilanes produce large quantities of HCl and require additional processing and handling steps.

The optical agent of this invention may be entrapped in void volumes created by a deliberately rapid condensation of silane. Said optical agents may include an organic or inorganic photoluminescent compound active in the UV region or an organic colorant active in the visible spectrum and may be employed singly or in combination, with the caveat that no portion of the optical agent is covalently reactive with the silane employed, unless and until the silane is polymerized and dried to produce a cured polyorganosilsesquioxane as hydrophobic, non-porous microparticles, in which case there may be covalent bonding between the optical agent and the polyorganosilsesquioxane. The relative reactivity of an optical agent to silane may be determined using conventional synthesis and analytical methods known in the art. For example, monofunctional silanes such as trimethyl methoxy silane, are preferentially used as test reagents for screening covalent reactivity of applicable optical agents in this context.

If an optical agent is first obtained in solid form, it may then be pre-formulated into a solution or dispersion using carrier solvents prior to addition into the PMSQ reaction vessel. Suitable carrier solvents may include, but are not limited to, silanes such as trimethoxymethylsilane, cyclomethicone, water, C₁-C₄ lower alcohols, diols, glycols, polyols, lower C₄-C₁₂ alkanes, chlorinated alkanes, toluene and xylene. Other additives may include organic acids for pH adjustment and surfactants or dispersants for improved processing and solubility when water is the solvent carrier. The optical agent is added at about 0.001% to about 1% weight by weight silane and charged to one or more reactant liquid phases that include the silane and/or water phase, prior to the initiation of polymer condensation. Said optical agent becomes a solute in the liquid phase or may be carried as an insoluble nanodispersion component in either reaction phase. A preferred nanodispersed system includes, but is not limited to a photoluminescent nanodiamond dispersed in the silane reactant or the water phase. Additional oils, surfactants and thickeners/suspending agents may optionally be used at 0.001% wt to about 1% wt based on optical agent wt % to enhance the dispersion stability or solubility of the optical agent in either reactant liquid phases.

Alkyltrialkoxysilanes, including but not limited to, methyltrimethoxysilane and methyltriethoxysilane become hydrolyzed in the presence of water at room temperature. Methyltrimethoxysilane is the preferred silane and used in all examples to follow. As the reaction proceeds and particles form, they precipitate out of solution, thus shifting the equilibrium towards formation of product. In the initial stage of the reaction, soluble intermediate methoxy derivatives of methylsilsesquioxane or oligosilicates, form and grow randomly in molecular weight until most of the methoxy groups are hydrolyzed, resulting in a milky white suspension of particles having a non-linear network structure of (MeSiO_{3/2}) with residual methoxy groups and a substantial amount of un-condensed hydroxyl groups present. Incorporation of the optical agent into the reaction mixture, prior to addition of the silane phase, results in the incorporation of the optical agent into the core of the precipitated microparticles. Residual optical agents which are not entrapped, and weakly adsorbed on the surface of the beads, are easily removed in subsequent washing steps. Not desiring to be bound by theory, it is believed that the optical agent becomes entrapped within the polymer microparticle as it forms. As such, adding the optical agent to an already precipitated reaction product has little utility in comparison to adding the optical agent prior to reaction initiation.

Hydrolysis of the silanes occurs spontaneously when silane is combined with any amount of water. The reaction can be catalyzed by the addition of acids or bases used to promote the hydrolysis reaction and facilitate the formation of the condensation product. Acid catalysts used include diluted HCl and acetic acid. Basic catalysts, such as aqueous ammonia, aqueous NaOH or triethanolamine, are preferably used to speed up the rate of reaction. When the hydrolysis occurs at moderate acidic condition, the particle size tends to be small. When at a strong basic condition, the particle size tends to be large because the coalescence of oligomer droplets is more likely to occur. In general, the particle size and distribution are determined by a few factors such as the catalyst used and its concentration, reaction temperature, mixing speed, interface tension and the feeding ratio of monomer/water. As the reaction is exothermic, a large amount of water is preferably used to absorb the released heat to better control the reaction temperature such that uniform particles are formed. An oil or surfactant can be used at 0.001% wt to about 2% by wt (based on silane wt %) to alter the surface tension between the oil phase and the water phase, where the reaction occurs to form spherical particles. The oil can be selected from silicone oil, mineral oil or synthetic oil. Mineral oil or silicone oil, especially low viscosity silicone oil is preferred. Surfactants can include the general class of anionic, amphoteric, non-ionic and cationic surfactants as selected from McCutchen's Emulsifiers and Detergents handbook (MC publishing Co. Glen Rock, N.J. USA). Anionic surfactants are preferred, including, but not limited to the C₈-C₁₆ alkyl sulfates, neutralized with counter ions of alkali metal, such as sodium or magnesium, or neutralized by amines, such as ammonia or triethanolamine. A water-soluble thickener may also be useful at about 0.001% wt to about 1% by wt (based on silane wt %) as a suspending agent to prevent the full-grown particles from coagulating with each other and warrant a narrow particle size distribution. The thickener/suspending agent may include, but is not limited to natural polymers like starches, xanthan gum, modified cellulosics, alginates, glucomannans, galactomannans and synthetic polymers like polyvinylpyrolidones, polyacrylates, polyacrylamides and mixtures thereof.

Preferred optical agents are non-ionic and more preferred optical agents are cationic in nature as it was discovered that anionic optical agents used in concert with anionic surfactants were not well entrapped in the growing polymer seed. Not wishing to be bound by theory, it is believed that anionic agents are not entrapped efficiently due to a coulombic repulsion that dominates in the early portion of the reaction. We propose that hydrolysis conditions using basic catalyst, designated as B⁺, promotes a fraction of soluble anionic reaction intermediate, possibly of the silanolate or silicic acid type, that may repel anionic optical agents while attracting cationic agents in this critical early reaction stage. Thus cationic optical agents are incorporated to a high degree in the polymer seed. Non-ionic optical agents are incorporated non-discriminately to a degree which correlates to their starting concentration. The reaction is about 90% complete after about 30 min at room temperature, but preferably allowed to react for an additional 8 hours. At this final stage, the degree of coulombic repulsion is near zero such that the final particle has very different electronic properties as compared to the intermediate silanol, RSi(OH)₃.

Subsequent processing, to remove impurities and excess optical agent which is not entrapped in the core of the microparticle, include repeated washing-soaking cycles with a large excess of DI water or other solvents, including the lower alcohols or mixtures thereof with water. The washing cycle may be in hot or cold, with the final number of cycles required varying according to the concentration of product in water, the temperature of the washes and on the final purity required. Typically between about 1 to about 8 wash cycles are essential to remove all optical agents which are not entrapped in the microparticle core. The preferred number of wash cycles is about four or less, particularly when at least one wash cycle is conducted at a temperature of between 25° C. and 100° C. The product is recovered through filtration as a wet cake containing typically up to about 40% water relative to its own weight. The wet product still contains residual —OH groups and can optionally be subsequently dispersed in the aqueous phase of a certain cosmetic preparations, even though the product is uncured. In this optional case, the optical agent is typically hydrophobic while the cosmetic preparation is essentially aqueous and not an emulsion. The wet polymer containing the entrapped optical agent is dried in a dehydration chamber such that water weight percent falls to about <1% by wt. Preferably, the drying step occurs at elevated temperatures of between about 100° C. to about 200° C., such that the dehydration process occurs rapidly, without degrading the integrity of the optical agent. Upon drying, the microparticles can be dispersed in an oil phase of a cosmetic preparation as the free hydroxyl groups and residual water has been removed and the final cured particle is essentially non-porous, fully cross linked and the particle surface essentially free of optical agent. The term “essentially free of optical agent” is defined as the point beyond which the surface of the particle after washing and curing is chemically indistinguishable from untreated PMSQ in regards to bulk surface properties, such as interfacial tension and zeta potential measurements.

Preferably, optical agents may include cationic pyrazoline compounds of the formula:

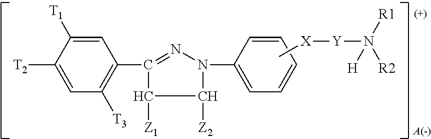

wherein:
X is O, SO₂, SO₂ NZ or a direct bond,
Y is an alkylene chain which may be interrupted by O, S or CONH,
Z is H or alkyl,
R₁ and R₂ are singly an alkyl, cycloalkyl or aralkyl radical or together with the adjacent nitrogen atom form a 5 to 7 membered N-heterocycle, which may optionally contain O, S or N as additional heteroatoms,

### T₁, T₂, T₃ are H, CH₃ or Cl,

Z₁ is H or alkyl,
Z₂ is H, alkyl or aryl, and
A is an anion of an organic acid.

Suitable alkyl radicals R₁ and R₃ are especially those having 1 to 4 carbon atoms, which may be substituted by halogen such as fluorine, chlorine and bromine hydroxyl groups, cyano groups, C₁-C₄-alkoxy groups, phenoxy groups, C₂-C₅-alkylcarbonyloxy groups or C₂-C₅-alkoxycarbonyloxy groups.

Suitable cycloalkyl radicals R₁ and R₃ are cyclopentyl and cyclohexyl radicals.

Suitable aralkyl radicals R₁ and R₃ are especially benzyl and phenylethyl radicals.

Suitable heterocyclic radicals which can be formed by R₁ and R₃ combining with the nitrogen atom are for example pyrrolidine, piperidine, imidazole, morpholine and thiomorpholine radicals.

Suitable alkyl radicals Z, Z₁ and Z₂ are especially unsubstituted alkyl radicals having 1 to 4 carbon atoms.

Suitable aryl radicals Z₂ are in particular phenyl radicals, which may be substituted by one or more halogen atoms, C₁-C₄ alkyl groups, C₁-C₄-alkoxy groups, cyano groups, carboxylic ester groups and carboxamide groups.

Useful alkylene radicals Y are especially those having 2 to 4 carbon atoms such as

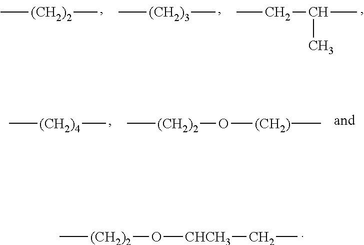

The anion A (−) may be an anion of a low molecular weight organic acid, examples being formate and lactate.

Most preferred pyrazoline compounds are those where

### X is SO₂,

### Y is —CH₂CH₂—, —CH₂CH₂CH₂—, —CH₂CH₂OCH₂CH₂,

R₁ and R₂ are each C₁-C₂-alkyl,
T₁-T₃ do not represent Cl or CH₃ at one and the same time,
A is lactate or formate or mixtures thereof.

For example, a preferred pyrazoline base compound has the formula

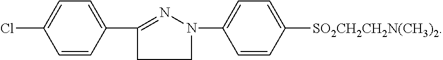

If an optical dye, photoluminescent nanodiamond or organic optical agent, as specified in this invention, is first obtained in solid form, they may then be pre-formulated into a solution or dispersion form using carrier solvents prior to addition into the PMSQ reaction vessel. Suitable carrier solvents may include, but are not limited to, silanes such as trimethoxymethylsilane, cyclomethicone, water, C₁-C₄, lower alcohols, diols, glycols, polyols, lower C₄-C₁₂ alkanes, chlorinated alkanes, toluene and xylene. Other additives may include organic acids for pH adjustment and surfactants or dispersants for improved processing and solubility when water is the solvent carrier.

Water and trimethoxyalkylsilane are the preferred carrier solvents for any optical or photoluminescent material used in this invention, additionally serving as reactants in the synthesis of PMSQ powder. Non-soluble solids, including photoluminescent nanodiamonds, may be stably dispersed into a carrier solvent by using mechanical or acoustical means only, such as a colloid mill or ultrasonic horn disperser.

Examples of optical agents that are of the preferred organic cationic photoluminescent type include, but are not limited to, Leucophor KCB Liquid, Hostalux ACK and Hostalux NR liq, all available from Clariant Corporation, Charlotte N.C. USA.

Other examples of optical agents are FD&C and D&C dyes. Preferably D&C Red 17, D&C Green 6, D&C Violet 2, D&C Yellow 11 and D&C Black 2.

Other examples of optical agents include commercially available nanodiamonds commonly known as “Nanodiamonds” (from Nanoblox Inc.) or “Carbon Based Quantum Dots” (Selah nanotechnologies) or “Crystalline Nanodiamond” or “Detonation Nanodiamond” or “Non-Detonation Nanodiamond” or “Carbon Nanoparticles”. The nanodiamonds are also commercially available with a number of different photoluminescent properties. The nanodiamonds are also commercially available with a number of different surface functionalities. The preferred class of nanodiamond is commonly referred to as ultrananocrystalline diamond, produced by detonation synthesis, with characteristic size of about 10 nm. Another preferred class of nanodiamond is commonly referred to as amorphous carbon.

In a particularly preferred feature of the invention, we have found that we can achieve a synergistic optical brightening effect, greater than the additive optical brightening effect, when the nonporous microparticles of polyorganosilsesquioxane contain as optical brighteners, nanodiamond particles combined with a second optical brightener, especially a second optical brightener, including the organic cationic photoluminescent compounds such as the pyrazoline cationic optical brighteners such as Leucophor KCB Liquid, Hostalux ACK and Hostalux NR liq. The weight ratio between the cationic optical brightener and the nanodiamond particles in the nonporous microparticles of polyorganosilsesquioxane ranges from 1:10 to 10:1, preferably 1:5 to 5:1 and more preferably 5:2.

In another particularly preferred feature we have found that we can achieve a synergistic optical effect for improving the appearance of wrinkled skin, greater than the additive optical effect when each component is applied alone to wrinkled skin, when the nonporous microparticles of polyorganosilsesquioxane contain as optical brighteners, nanodiamond particles combined with an optical dye such as D & C Green No. 6 or D & C Red No. 17. The weight ratio between the nanodiamond particles and the optical dye ranges from 1:10 to 10:1, preferably 1:5 to 5:1, and more preferably about 1:1.

In another particularly preferred feature, the nonporous microparticles of polyorganosilsesquioxane containing as optical brighteners, both nanodiamond particles and a second optical brightener, such as a cationic pyrazoline as discussed hereinabove, are combined with other nonporous microparticles of polyorganosilsesquioxane containing optical dyes, such as D & C Green No. 6 or D and C Red No. 17. The combination of both types of non-porous microparticles of polyorganosilsesquioxane blended together in equal parts by weight in a personal care, cosmetic or pharmaceutical composition has proved especially effective in improving the appearance of wrinkled skin.

The hydrophobic PMSQ powder with entrapped optical agent is uniquely useful for formulating cosmetic or pharmaceutical products with the traditional feel and character of PMSQ microparticles, but with novel optical properties that allow for a refinement of color appearance or softness appearance of under lighting conditions, for example, containing a UV component. The cosmetic compositions are not particularly restricted to any format of, for example, gel, lotion, cream, foundation, loose powder, press powder, stick, soap and paste. Mention may be made of any additives usually delivered to the skin, such as fillers and/or pearlescent agents, anti-foam agents, antioxidants, opacifiers, fragrances, preserving agents, cosmetic or pharmaceutical active agents, sunscreens, antiperspirant agents and self-tanning agents, each in an effective amount to accomplish its respective functions.

Other applications for the silsesquioxane with optical agent of the present invention include, but are not limited to:
1) Inclusion as a component filler in fibers for producing textiles with permanent brightening attributes
2) Electronic and optical displays for attenuating the conversion of solar energy into electricity and attenuating the refractive layer in display screens, including, but not limited to LCD displays.
3) Inclusion as a component in paints, inks and coatings for producing colors and coatings with permanent brightening and novel color attributes, preferably including ink jet printing applications.

The inventive micronized particles with entrapped optical agent as delineated in this invention may also serve to scatter light in applications as per the Mie theory, also called Lorenz-Mie theory or Lorenz-Mie-Debye theory, whereby these theories are a complete analytical solution of Maxwell's equations for the scattering of electromagnetic radiation by spherical particles (also called Mie scattering). Lorenz-Mie theory is named after its independent developers, German physicist Gustav Mie and Danish physicist Ludvig Lorenz who developed the theory of electromagnetic plane wave scattering by a dielectric sphere. Independent Rayleigh scattering may also occur in cases where nanodiamond is present in the core of the PMSQ particle.

### BRIEF DESCRIPTION OF THE DRAWINGS

FIG. 1 is a drawing illustrating a micronized particle of PMSQ in which is contained an entrapped optical agent, such as nanoparticles of diamond, and in which, the path of the incident light beamed to the surface of the micronized particle of PMSQ is illustrated as well as the Mie and Rayleigh scattering of light and photoluminescence that results when the incident light penetrates the PMSQ particle to reach the optical agent, which then emits diffused and altered light from the micronized particle of PMSQ.

FIG. 2 is a drawing illustrating the micronized particle of PMSQ according to FIG. 1 applied in a cosmetic composition to the surface of wrinkled skin showing how the appearance of the wrinkled skin is altered by the visible diffused and altered light, emitted by the entrapped optical agent from the microparticles of PMSQ to the eye of the viewer.

### DETAILED DESCRIPTION OF THE DRAWINGS

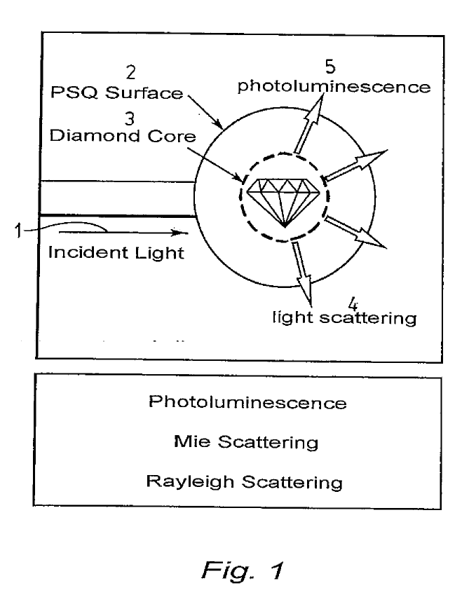

According to FIG. 1, incident light (1) passes through the light permeable surface of the micronized particle (2) of PMSQ, and reaches the surface of the optical agent (3) e.g. nanoparticles of diamond. Once the light reaches the optical agent, light scattering takes place and the scattered light (4) emitted by the optical agent passes back through the surface of the micronized particle of PMSQ creating photoluminescence (5) which is visible altered and diffused light.

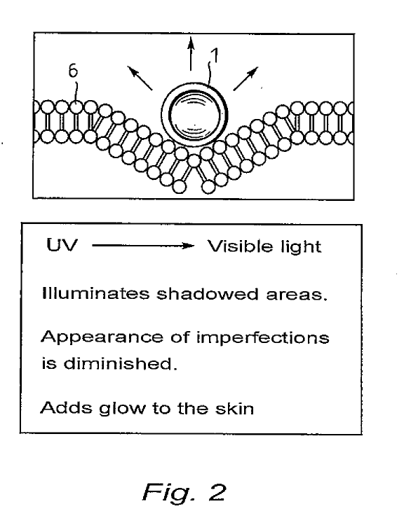

According to FIG. 2, the micronized particle of PMSQ (1) is part of a cosmetic formulation, topically applied to wrinkled skin (6). The photoluminescence (5) which is emitted by the optical agent as visible altered and diffused light so that one who is looking at the wrinkled skin coated with the cosmetic formulation will view the skin with diminished perception of wrinkles and other skin imperfections, will view the skin with an added glow and with a more desirable color, and will see areas of the skin normally hidden by shadows with a greater degree of illumination.

### EXAMPLES

The invention is illustrated in the examples below, which are not intended to be restrictive. Examples 1 and 2 describe the preparation of PMSQ powder without optical agent as comparative examples under acidic and basic conditions. Example 3. uses an anionic fluorophore in the attempt to post-fix it to example 2. Example 4 uses a cationic fluorophore in the attempt to post-fix it to example 2. Example 5 prepares an uncured PMSQ as a comparative example without optical agent. Example 6 attempts to post-fix an anionic fluorophore to the uncured particle of example 5 after the particle is isolated. Examples 7-10 are to be under the scope of invention. Example 11 is a cosmetic composition under the scope of invention.

### Example 1

### Preparation of Hydrophobic PMSQ Powder Under Acidic Condition

To a reactor containing 100 parts of water and 0.02 part of xanthan gum at 15° C. were added 28 parts of methyltrimethoxysilane and 2 parts of cyclopentasiloxane. The mixture was mixed very slowly at 5 rpm at 25-29° C. and pH-3.4 for 24 hours. A particulate product partially precipitated to the bottom in the reactor was collected by centrifuging and added to a strong base solution of 20 parts of water and 0.4 part of NaOH to allow for a post-hydration reaction at room temperature for 24 hours. The product was washed at least three times or until the pH was neutral by using water followed by centrifuging. The resulting particles had a spherical shape and the particle size was between 1-4 microns with a mean value of 2.5 microns. The product was a wet powder and was then cured at 185° C. for a duration of 4 hours to a dry state of <0.2% water. The particles under short wave UV light had no observable fluorescent property.

### Example 2

### Preparation of Hydrophobic PMSQ Powder Under Basic Condition

To a reactor containing 100 parts of water, 0.06 part of xanthan gum and 0.07 part of 28% aqueous ammonia at 15° C. were added 28 parts of methyltrimethoxysilane and 3 parts of isohexadecane. The mixture was mixed at 300 rpm at 15-22° C. and pH 9.5-10.0 for 3 hours and kept in the reactor for 24 hours without mixing; yielding a particulate product that was precipitated to the bottom in the reactor. The rest of the procedures were essentially the same as described in Example 1. The resulting particles had a spherical shape and the particle size was between 5-13 microns with a mean value of 10 microns. The product was a wet powder and was then cured at 185° C. for a duration of 4 hours to a dry state of <0.2% water. The particles under short wave UV light had no observable fluorescent property.

### Example 3

### Failed Attempt to Post-Fix an Anionic Fluorophore to the Particle of Example 2

To a reactor containing 10 g of the control product of Example 2, 10 g of Leucophor BSB (an anionic fluorophore in water from Clariant Corporation, Charlotte N.C. USA) was added to form a slurry and heated to 65° C. for a duration of 2 hours with stirring. The slurry was collected on a filter paper and rinsed with a large excess of water until the concentrated filtrate was uncolored under short wave UV light. The product was removed from the filter paper and dried to <0.2% water by wt. The particles under short wave UV light were not significantly different in fluorescent property from the control example 2.

### Example 4

### Failed Attempt to Post-Fix a Cationic Fluorophore to the Particle of Example 2

The same general reaction as described in Example 3 was again performed, except this time using a cationic fluorophore, Hostalux NR liq (Clariant Corporation). The particles under short wave UV light were not significantly different in fluorescent property from the control Example 2.

### Example 5

### Preparation of Uncured, Hydrophilic PMSQ Powder Control Under Basic Condition

To a reactor containing 100 parts of water, 0.12 parts of hydroxyethylcellulose and 0.18 parts of 28% aqueous ammonia at 15° C. were added 28 parts of methyltrimethoxysilane and 3 parts of isohexadecane. The mixture was mixed at 5 rpm at 15-28° C. and pH 9.5-10.0 for 3 hours and kept in the reactor for 24 hours without mixing, yielding a particulate product that was precipitated to the bottom in the reactor. The particulate was collected and added to a strong base solution of 20 parts of water and 0.4 part of NaOH to allow for a post-hydration reaction at room temperature for 24 hours. The product was washed three times such that the pH was neutral by using water followed by centrifuging. The resulting particles had a spherical shape and the particle size was between 1-4 microns with a mean value of 2.5 microns. The product was a hydrated powder in an uncured state.

### Example 6

### Attempt to Post-Fix an Anionic Fluorophore to Uncured Particle of Example 5

To a reactor containing 10 g of the control product of Example 5, 10 g of Leucophor BSB was added, forming a paste-like slurry, and then heated to 65° C. for a duration of 2 hours with stirring. The slurry was collected on a filter paper and rinsed with a large excess of water until the concentrated filtrate was uncolored under short wave UV light. The product was removed from the filter paper and cured at 185° C. for a duration of 2 hours to <0.2% water by wt. The particles under short wave UV light had a only slightly increased fluorescent property compared to the control example 5, thus indicating it as a poor substrate choice for the post fixing of an optical agent.

### Example 7

### Synthesis of PMSQ Microparticles with Entrapped Nanodiamond and Cationic Fluorophore

To a reactor containing 20 kg of water, 50 g Hostalux NR liq, 0.06 parts of xanthan gum, 0.1 parts sodium dodecyl sulfate and 0.07 parts of 28% aqueous ammonia at 15° C. were added the premixed silane phase of 28 parts of methyltrimethoxysilane and 20 g Nanodiamond-EDA (Nanoblox Inc.) The mixture was mixed at 300 rpm at 15-22° C. and pH 9.5-10.0 for 3 hours and then kept in the reactor for 24 hours without mixing; yielding a particulate product that was precipitated to the bottom in the reactor. The liquid top layer was decanted and an excess volume of water was added and the product was redispersed by mixing for ½ hour. The cycle of decanting and washing was repeated 5 additional times for a total of six washes. The resulting particles had a spherical shape and the particle size was between 5-13 microns with a mean value of 10 microns. The product was a wet powder and was then cured at 185° C. for a duration of 4 hours to a dry state of <0.2% water. The product under short wave UV light had intense fluorescent activity in the blue and green region of the electromagnetic spectrum. Fluorescence intensity was 40% greater when compared with microparticles containing the same amount of fluorophore (Example 8) or nanodiamond alone (Example 9). The interfacial properties of the particle were identical with the control particle from example 1 in dispersion tests in both water and cyclotetrasiloxane.

### Example 8

### Synthesis of PMSQ Microparticles with Entrapped Cationic Fluorophore

The same reaction, washing and drying steps were followed exactly as per Example 7, except the nanodiamond component was not used in any aspect. The product had intense fluorescent activity, but was 40% less fluorescent than the microparticles of Example 7.

### Example 9

### Synthesis of PMSQ Microparticles with Entrapped Nanodiamond

The same reaction, washing and drying steps were followed exactly as per Example 7, except the Hostalux NR liq component was not used. The product had slight fluorescent activity of only 10% compared to the product of Example 7.

Review of Examples 7-9 indicated an unexpected synergism between entrapped nanodiamond and fluorophore, resulting in an increase of fluorescence intensity of up to 40%.

### Example 10

### Preparation of Hydrophobic PMSQ Powder Under Basic Conditions with Non-Ionic Optical Agent

To a reactor containing 100 parts of water, 0.06 parts of xanthan gum, 0.1 parts sodium dodecyl sulfate and 0.07 parts of 28% aqueous ammonia at 15° C. were added the premixed silane phase of 28 parts of methyltrimethoxysilane and 2.8 parts D&C Green No 6. The mixture was mixed at 300 rpm at 15-22° C. and pH 9.5-10.0 for 3 hours and then kept in the reactor for 24 hours without mixing; yielding a colored particulate product that was precipitated to the bottom in the reactor. The liquid top layer was decanted and an excess volume of water was added and the product was redispersed by mixing for ½ hour. The cycle of decanting and washing was repeated 1 additional time for a total of two washes. The resulting particles had a spherical shape and the particle size was between 5-13 microns with a mean value of 10 microns. The product was a wet powder and was then cured at 185° C. for a duration of 2 hours to a dry state of <0.2% water. The product under short wave UV light had no fluorescent activity, but was a cool green color, appropriate for use as a colorant in cosmetic applications to cover the redness caused by aged or mottled skin or to tone the skin to offset the symptoms of rosacea.

### Example 11

### Cosmetic Powder

The powder of Examples 7 and 10 were blended together in equal parts and used directly as a mineral type cover powder on 5 subjects complaining of unevenly toned skin, wrinkled skin. All 5 test subjects instantly declared they felt their skin was brighter and more evenly toned after one application.

### Example 12

### Cosmetic Lotion

To 95% of an oil-in-water skin lotion was added 5% of the powder from Example 7 and admixed for 15 minutes. Likewise, a control lotion was created with the non-fluorescent powder of Example 1. The cosmetic lotion with the powder of Example 7 had intense fluorescent activity under short wave UV, while the control lotion using the powder of Example 1 had no fluorescent activity change compared to the control without any admixed powder.

We claim:

1. A non-porous microparticle of polyorganosilsesquioxane containing an entrapped optical agent whereby the surface of the particle is essentially free of optical agents.

2. The non-porous microparticle defined in claim 1, wherein said particle is at least 0.1 micron in size, but below 100 microns in size.

3. The non-porous microparticle defined claim 2, wherein said particle is one of a plurality of said particles essentially monodispersed in size with an average particle size between at least 1 micron to about 10 microns in size and at least 90% by weight of the particles fall within this size range.

4. The non-porous microparticle defined in claim 1, wherein said optical agent is active in the visible or UV electromagnetic spectrum and selected from the group consisting of D&C dyes, FD&C dyes, photoluminescent nanodiamonds, photoluminescent amorphous carbon nanoparticles and fluorescent dyes.

5. The non-porous microparticle defined in claim 4, wherein said optical agent is nonionic or cationic.

6. The non-porous microparticle defined in claim 4, wherein said fluorescent dye is capable of absorption in the UV electromagnetic spectra and provides emission in the blue-green region of the visible electromagnetic spectrum.

7. The non-porous microparticle defined in claim 6, wherein said fluorescent dye is cationic.

8. The non-porous microparticle defined in claim 7, wherein said fluorescent dye is a pyrazoline dye.

9. The non-porous microparticle defined in claim 4 wherein said optical agent is a nonionic D&C dye.

10. The non-porous microparticle defined in claim 4, wherein said optical agent is a photoluminescent nanodiamond or photoluminescent amorphous carbon particle made from detonation or non-detonation methods.

11. The non-porous microparticle defined in claim 1, wherein said optical agent is a mixture comprised of more than one ingredient active in the visible or UV electromagnetic spectrum and selected from the group consisting of D&C dyes, FD&C dyes, photoluminescent nanodiamonds, photoluminescent amorphous carbon nanoparticles and fluorescent dyes.

12. The non-porous microparticle defined in claim 11, wherein the optical agent is comprised of a mixture of nonionic and cationic ingredients.

13. The non-porous microparticle defined in claim 11, wherein said optical agent is a synergistic combination of D&C Green No 6 and photoluminescent nanodiamond which provide unique optical properties not present in microparticles containing green dye or nanodiamond alone.

14. The non-porous microparticle defined in claim 11, wherein said optical agent is a synergistic combination of D&C Red No 17 and photoluminescent nanodiamond which provide unique optical properties which are not present in microparticles containing red dye or nanodiamond alone.

15. The non-porous microparticle defined in claim 11, wherein said optical agent is a synergistic combination of photoluminescent nanodiamond and cationic fluorophore, such that the combination of optical agents broadens the emission spectrum and increases the emission intensity when compared to microparticles containing an equivalent amount of nanodiamond or cationic fluorophore alone.

16. The non-porous microparticle defined in claim 1, wherein the microparticles can convert light of higher energy to light of lower energy via photo luminescence, fluorescence and/or quantum confinement.

17. A personal care, cosmetic or pharmaceutical composition comprising about 0.1% to about 75% by weight of a plurality of non-porous microparticles of polyalkylsilsesquioxane containing an entrapped optical agent, whereby the surface of the plurality of microparticles is essentially free of optical agent, wherein the alkyl group in said polyalkylsilsesquioxane is selected from methyl, ethyl, propyl, butyl or phenyl radicals, either singly or in any combination thereof in combination with a cosmetically or pharmaceutically acceptable inert carrier or diluent.

18. The personal care, cosmetic or pharmaceutical composition defined in claim 17, selected from the group consisting of skin creams; eye creams; skin primers; wrinkle correctors; lotions; sunscreen lotions; after-sun lotions; shampoos; body rinses; bath gels; hair fixatives; hair conditioners, hair serums; nail polishes; soaps; hair color solutions; mascaras; eye shadows; eye liners; lipsticks; lip glosses; foundation liquids and loose or compressed powders; tooth pastes; oral rinses and in pharmaceutical preparations requiring an encapsulated fluorescent topical indicator for delivery purposes.

19. The personal care, cosmetic, or pharmaceutical composition defined in claim 18 used to reduce the appearance of skin imperfections or alter the appearance of the tone/hue of the skin, nails or hair.

20. The personal care, cosmetic or pharmaceutical composition defined in claim 17, wherein said plurality of microparticles reduces the perception of skin imperfections by one or more mechanisms including: enhanced soft focus scattering of light (via Mie scattering and/or Rayleigh scattering (the latter in compositions where nanodiamond is present)); imparting a color tone/hue on the skin; and/or by converting light of higher energy to light of lower energy when applied to the skin surface.

21. A method of reducing the appearance of imperfections in the skin of a subject in need of said treatment, wherein the imperfection is selected from the group consisting of mild scars, redness, wrinkles, shadows, discolorations and age spots, which comprises the step of topically applying to the skin of the subject, an effective amount of the personal care, cosmetic or pharmaceutical composition defined in claim 17.

22. A method of altering the appearance of skin or hair of a subject in need of said treatment comprising the step of contacting said skin or hair of said subject with a personal care composition comprising an effective amount of the microparticles defined in claim 4.

23. A process to produce a plurality of non-porous microparticles of polyorganosilsesquioxane containing an entrapped optical agent, whereby the surface of the plurality of microparticles is essentially free of optical agent, which comprises the steps of:
a) hydrolyzing and condensing an alkyltrialkoxysilane in water in the presence of about 0.001% to about 2% by weight of optical agent, with about 0% to about 1% by weight suspending agent, with about 0% to about 2% by weight a surface tension modifier and an acid or base catalyst to form spherical particles;
b) purifying the spherical particles by washing them from about 1 to up to about 10 times and;
c) optionally, drying the particles at elevated temperatures to cure them to a highly cross-linked state; wherein, weight % is in reference to the initial weight of silane charged and; the alkyl groups in said alkyltrialkoxysilane can be the same or different, selected from methyl, ethyl, propyl, butyl or phenyl radicals.

24. The process defined in claim 23, wherein each of said plurality of particles is at least about 0.1 micron in size, but below about 100 microns in size and the particles are essentially spherical in shape.

25. The process defined in claim 24, wherein each of said plurality of particles is essentially monodispersed in size with an average particle size between at least about 1 micron to about 10 microns in size and at least 90% by weight of the particles falling within this size range.

26. The process defined in claim 23, wherein the alkyltrialkoxysilane is methyltrimethoxysilane to provide polymethylsilsesquioxane as the polyorganosilsesquioxane.

27. A method of altering the appearance of synthetic fibers, plastic resins, paints, inks and coatings for producing colors and coatings to impart permanent brightening and novel color attributes, which comprises the step of incorporating as a filler in said synthetic fibers, plastic resins, paints, inks and coatings, a plurality of the particles defined in claim 1 in an effective amount to alter the appearance of the synthetic fibers, plastic resins, paints, inks and coatings for producing colors and coatings to impart permanent brightening and novel color attributes.
